(19) United States
(12) Patent Application Publication
(10) Pub. No.: US 2026/0054051 A1
(43) Pub. Date: Feb. 26, 2026

(54) DESCENDING AORTA AND VENA CAVA BLOOD PUMPS

(71) Applicant: Shifamed Holdings, LLC, Campbell, CA (US)

(72) Inventors: Janine ROBINSON, Half Moon Bay, CA (US); Daniel HILDEBRAND, Santa Cruz, CA (US); Michael CALOMENI, San Jose, CA (US); Veronica NEIMAN, San Jose, CA (US); Tom SAUL, Portland, OR (US); Robert EDESESS, Campbell, CA (US)

(21) Appl. No.: 19/302,496

(22) Filed: Aug. 18, 2025

Related U.S. Application Data
(63) Continuation of application No. 17/784,758, now Pat. No. 12,409,310.
(60) Provisional application No. 62/946,927, filed on Dec. 11, 2019.
(60) Provisional application No. 62/951,519, filed on Dec. 20, 2019.

(51) Int. Cl. A61M 60/122; A61M 60/237; A61M 60/808

(52) U.S. Cl. CPC A61M 60/122; A61M 60/808; A61M 60/237

(57) ABSTRACT

Methods and devices for supporting circulation. The methods may include positioning a blood pump in the arterial vasculature or the venous vasculature. The methods may include positioning a pump portion of the blood pump in a descending aorta, an inferior vena cava, a renal artery, and/or a renal vein. The methods include delivering a pump portion of a blood pump to a target location and rotating one or more impellers to move blood through the pump portion.

### CLAIM OF PRIORITY

This application is a continuation of U.S. Application No. Ser. No. 17/784,758, filed Jun. 13, 2022, which is a 371 of PCT/US2020/064586, filed Dec. 11, 2020, which claims the benefit of priority of U.S. Provisional Application No. 62/946,927, filed Dec. 11, 2019, and U.S. Provisional Application No. 62/951,519, filed Dec. 20, 2019, the complete disclosures of which are incorporated by reference herein for all purposes.

### INCORPORATION BY REFERENCE

All publications and patent applications mentioned in this specification are herein incorporated by reference to the same extent as if each individual publication or patent application was specifically and individually indicated to be incorporated by reference.

The following publications are incorporated by reference herein for all purposes: WO2018/226991A1, WO2019/094963 A1, WO2019/152875A1, WO2020/028537A1, WO2020/073047A1, WO2018/226991A1, WO2019/094963A1, WO2019/152875A1, U.S. Pat. No. 9,572,915, and US 2017/0100527.

### BACKGROUND

Descriptions have been presented that attempt to increase renal artery and kidney perfusion using a blood pump positioned in a descending aorta. One or more aspects of those descriptions, however, may have deficiencies that can be addressed by catheter-based blood pumps and methods of placement and use that are set forth herein.

Additionally, descriptions have been presented that attempt to reduce blood pressure within one or more renal vein by placing a blood pump in a vena cava and pumping blood away from the region. One or more aspects of those descriptions, however, may have deficiencies that can be addressed by catheter-based blood pumps and methods of placement and use that are set forth herein.

### SUMMARY OF THE DISCLOSURE

One aspect of the disclosure is a method of supporting circulation in a descending aorta of a subject. The method may include advancing an expandable pump portion of a blood pump into a descending aorta of a subject, the pump portion comprising an expandable blood conduit between a pump inflow and a pump outflow, the pump portion further including first and second expandable impellers at least partially within the blood conduit. The method may further include expanding the expandable blood conduit to an expanded and deployed configuration within the descending aorta. The method may further include expanding the first expandable impeller into an expanded configuration at least partially within the blood conduit and expanding the second expandable impeller into an expanded configuration at least partially within the blood conduit, and rotating the first and second expandable impellers to thereby move blood into the pump, through the blood conduit, and out of the pump. The rotating step may move blood through the blood conduit at a rate of at least 3.5 L/min.

In this aspect, the rotating step may cause the pump outflow to include a radial flow component.

In this aspect, moving blood out of the pump outflow may perfuse at least one renal artery.

In this aspect, expanding the expandable blood conduit may comprise expanding the expandable blood conduit to a deployed configuration within the descending aorta such that the pump outflow is upstream or aligned with a renal artery, and wherein the outflow is at least partially directed radially into the renal artery due to the position of the blood conduit in the descending aorta.

In this aspect, expanding the expandable blood conduit to a deployed configuration within the descending aorta may comprise expanding the expandable blood conduit to a deployed configuration near a renal artery such that the pump outflow perfuses at least one renal artery.

In this aspect, expanding the second expandable impeller may comprise expanding the second expandable impeller into an expanded configuration such that at least a portion of the second expandable impeller is extending beyond a proximal end of the blood conduit, and wherein the outflow may have a radial flow component due at least partially to the portion of the second expandable impeller that is extending beyond a proximal end of the blood conduit.

In this aspect, expanding the expandable blood conduit within the descending aorta may comprise expanding a distal scaffold to an expandable configuration that provides radial support to the blood conduit at the location of the first impeller. Expanding the expandable blood conduit within the descending aorta may comprise expanding a proximal scaffold to an expandable configuration that provides radial support to the blood conduit at the location of the second impeller.

In this aspect, expanding the expandable blood conduit may comprise expanding a central region of the blood conduit that is between the first and second impellers, wherein the central region may be more flexible than regions of the blood conduit that surround the first and second impellers.

In this aspect, expanding the expandable blood conduit to a deployed configuration within the descending aorta may comprise expanding the expandable blood conduit to a deployed configuration within the descending thoracic aorta.

One aspect of this disclosure is a method of supporting circulation in a descending aorta of a subject. The method may include advancing an expandable pump portion of a blood pump into a descending aorta of a subject, the pump portion including a distal impeller and a proximal impeller; expanding a distal expandable blood conduit into an expanded configuration aligned with or upstream to a renal artery; expanding the distal impeller to an expanded configuration at least partially within the distal expandable blood conduit; expanding a proximal expandable blood conduit into an expanded configuration downstream from the renal artery; expanding the proximal impeller to an expanded configuration at least partially within the proximal expandable blood conduit; rotating the distal impeller to move blood into a distal end of the distal expandable blood conduit, through the distal expandable blood conduit, and out of a proximal end of the distal expandable blood conduit; and rotating the proximal impeller to move blood through the proximal expandable blood conduit.

In this aspect, rotating the distal impeller may cause blood to move out of the proximal end of the distal expandable blood conduit and perfuse the renal artery, optionally also perfusing a second renal artery.

In this aspect, rotating distal and proximal impellers may move blood past the distal and proximal impellers in an antegrade direction.

In this aspect, rotating the distal impeller may move blood past the distal impeller in an antegrade direction, and rotating the proximal impeller may move blood past the proximal impeller in a retrograde direction toward the renal artery.

In this aspect, rotating the distal and proximal impellers may be performed discontinuously and in a manner that is related to one or more aspects of the subject's cardiac cycle.

In this aspect, rotating the distal and proximal impellers may occur during at least a portion of systole, and wherein the distal and proximal impellers may not be rotated during at least a portion of diastole, optionally during any of diastole.

In this aspect, rotating the proximal impeller may move blood into a distal end of the proximal expandable blood conduit, through the proximal expandable blood conduit, and out of a proximal end of the proximal expandable blood conduit.

In this aspect, expanding a distal expandable blood conduit into an expanded configuration aligned with or upstream to a renal artery may comprise expanding a distal expandable scaffold.

In this aspect, expanding a proximal expandable blood conduit may comprise expanding a proximal expandable scaffold.

In this aspect, rotating the distal and proximal impellers may be performed by rotating a common drive mechanism to which the distal and proximal impellers are in rotational communication.

In this aspect, the method may further comprise causing blood to flow radially outward from a proximal end of the distal blood conduit.

One aspect of the disclosure is a method of supporting circulation in proximity to renal veins. The method may include advancing an expandable pump portion of a blood pump into an inferior vena cava (“IVC”) of a subject, the pump portion comprising an expandable blood conduit between a proximal end and a distal end, the pump portion further including first and second expandable impellers; expanding the expandable blood conduit to a deployed configuration within the IVC; expanding the first expandable impeller into an expanded configuration at least partially within the blood conduit; expanding the second expandable impeller into an expanded configuration at least partially within the blood conduit; and rotating the first and second expandable impellers to move blood into the blood conduit, through the blood conduit, and out of the blood conduit. This aspect may additionally include any other suitable method step described herein.

### BRIEF DESCRIPTION OF THE DRAWINGS

FIG. 1 is an example of an expandable pump portion secured in a descending aorta.

FIG. 2 is an example of an expandable pump portion with one or more inflatable anchors secured in a descending aorta.

FIG. 3 is an example of an expandable pump portion secured in a descending aorta.

FIG. 4 is an example of an expandable pump portion secured in a descending aorta.

FIG. 5 is an example of an expandable pump portion secured in an inferior vena cava.

### DETAILED DESCRIPTION

The disclosure is related to catheter blood pumps that may be placed in one or more of the following locations: a descending aorta, an inferior vena cava (“IVC”), a renal artery, or a renal vein. The blood pumps herein may include pump portions that include one or more impellers within a blood conduit, wherein the one or more impellers are sized and configured to move blood through the blood conduit when rotated.

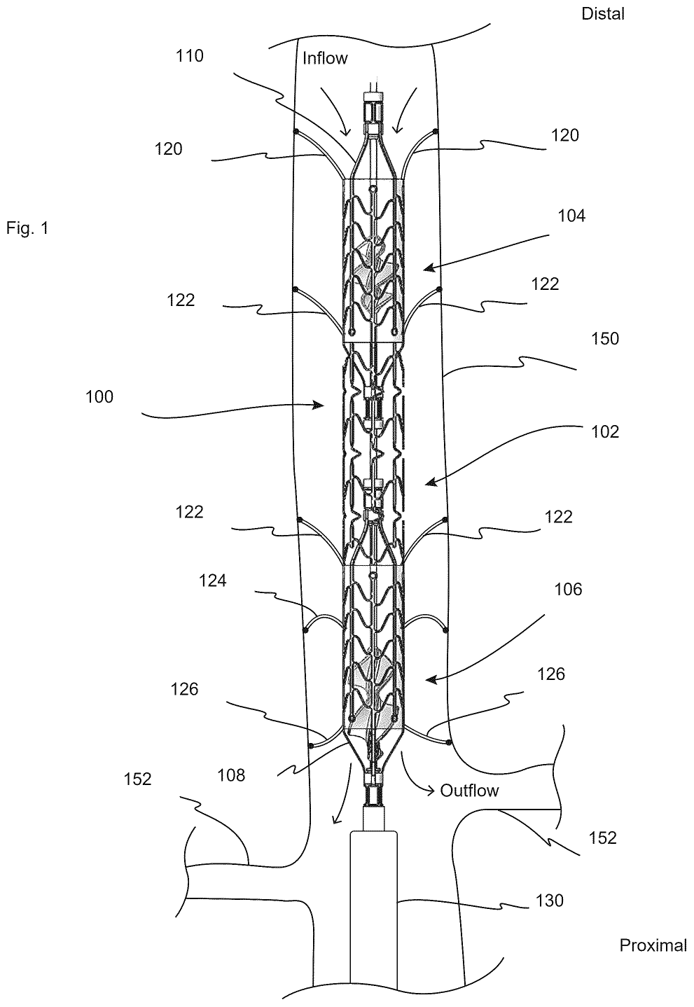

One aspect of the disclosure is related to intravascular multiple impeller blood pumps that include pump portions that are sized and configured for placement in the descending aorta, optionally within the abdominal aorta, such as is shown in the examples in FIGS. 1-3. The intravascular multiple impeller blood pumps may be configured for cardiovascular and circulatory support. The entirety of U.S. Pat. No. 9,572,915, including the background section thereof, is fully incorporated by reference herein in this context. For example, intravascular multiple impeller blood pumps herein may be used in cardiovascular support, and may be used to facilitate perfusion of targeted vessel and/or organs, such as the kidneys.

One aspect of the disclosure is related to intravascular multiple impeller blood pumps that include pump portions that are sized and configured for placement in an inferior vena cava (“IVC”). The background section of US 2017/0100527 is fully incorporated by reference herein in this context. For example, intravascular multiple impeller blood pumps herein may be placed in the vicinity of a junction of an IVC and one or more renal veins, and may be used to facilitate blood flow in the venous vasculature, optionally to reduce renal venous pressure. Any of the disclosure in US 2017/0100527 related to delivery of a blood pump to a target location near the renal veins is incorporated by reference herein for all purposes, including any methods herein that include delivering a blood pump to a target location and/or expanding a blood pump at a target location. The disclosure in WO2018/226991A1, WO2019/094963 A1, WO2019/152875A1, WO2020/028537A1, WO2020/073047A1 may include exemplary multiple impeller blood pumps, any of which may be delivered, deployed, and operated to move fluid therethrough for cardiovascular and circulatory support according to any of the methods herein. In any of the relevant methods herein, a multiple impeller blood pump may be delivered to a location in a patient's descending aorta, such as in the abdominal aorta.

FIGS. 1-3 illustrate exemplary pump portions of multiple impeller blood pumps deployed in expanded configurations in the descending aorta, with the impellers shown expanded and rotating to pump blood through the blood conduit of the pump portion of the blood pump. The deployed and expanded pump portions shown in FIGS. 1-3 may be similar to the pump portions shown and described in WO2018/226991A1, WO2019/094963 A1, WO2019/152875A1, WO2020/028537A1, WO2020/073047A1, and any suitable disclosure in any of these publications incorporated fully by reference herein for all purposes may be incorporated by reference into the description of FIGS. 1-3 herein.

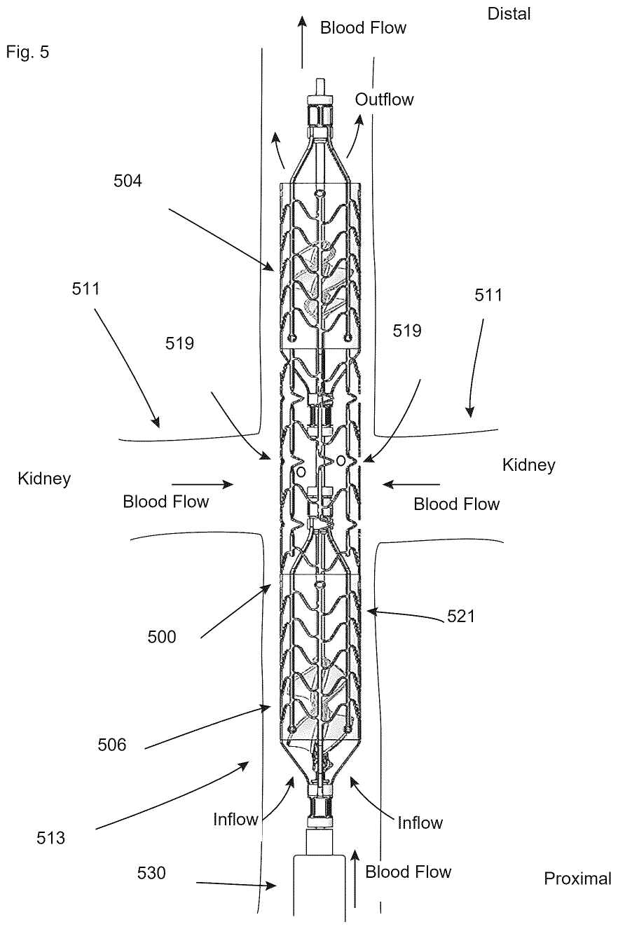

An exemplary advantage of the blood pumps and methods shown in FIGS. 1-3 is that the multiple impeller pump may be able to achieve higher flow rates than some existing single impeller blood pumps that are positioned in the descending aorta. The advantages of relatively low-profile and higher flow rate expandable blood pumps may be described in more detail in WO2018/226991A1, WO2019/094963 A1, WO2019/152875A1, WO2020/028537A1, WO2020/073047A1, and similarly apply to the relatively low-profile and higher flow rate blood pumps positioned for use in the descending aorta, such as those shown in FIGS. 1-3, or for use in a vena cava, such as shown in FIG. 5. For example, without limitation, the pump portions of the blood pumps herein may be sized and configured such that they can be introduced via a 10F introducer, while being able to achieve flow rates of at least 3.5 L/min when expanded and activated, which may be higher than some existing pump portions that are designed for placement in the descending aorta or vena cava.

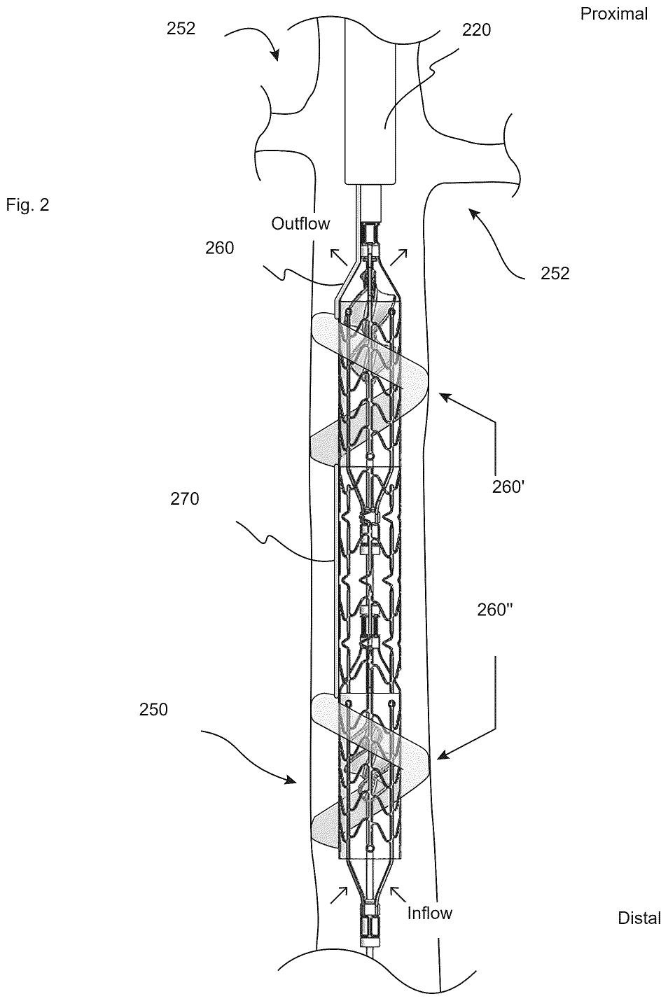

Any of the blood pumps herein may include one or more anchors (which may also be referred to herein as stabilizing members) that are sized and configured to help stabilize a pump portion of the blood pump with respect to the descending aorta or IVC, examples of which are shown in FIGS. 1 and 2. To anchor or stabilize as used in this context refers generally to being anchored in the aorta (or IVC) to prevent axial migration of the pump relative to the aorta, although some minimal movement may still occur, such as due to slight deformation of one or more anchors when the pump is in use. FIG. 1 illustrates an exemplary pump portion 100 of a blood pump in an expanded configuration stabilized in place in descending aorta 150 and generally proximate to renal arteries 152, with most or all of pump portion 100 upstream the renal arteries. Pump portion 100 includes one or more anchors or stabilizing members (120, 122, 124, 126) that in their expanded configurations extends radially outward and optionally also distally from an expandable blood conduit or shroud 102 of the blood pump, as shown. Pump portion 100 may include one or more anchors, and any of those shown in FIG. 1 may be optional. For example, the pump may only have one or more distal stabilizing members 120 extending from a distal end or distal region of the expandable blood conduit, as shown. It may be desirable to have anchors at one or more ends or end regions of the pump, such as anchors 120 and anchors 126, and/or stabilizing members that are disposed axially between the fluid conduit ends, such as one or more of stabilizing members 122 or stabilizing members 124.

Any of the anchors or stabilizing members herein may comprise one or more materials such that the anchor is adapted to self-expand to an at least partially expanded configuration. For example, any of the anchors or stabilizing members herein may be adapted to self-expand after a sheath, and example of which is shown in FIG. 1 as sheath 130, is retracted proximally to allow the anchor(s) to expand. Any of the anchors herein may be re-sheathed before or after final pump deployment and activation if pump repositioning is needed, or if the pump is to be re-sheathed and removed from the patient, such as with sheath 130 shown in FIG. 1. Sheath 130 is adapted to be axially movable relative to the catheter and pump portions that are shown to deploy and sheath the expandable pump portion, which may be described in more detail in WO2018/226991A1, WO2019/094963 A1, WO2019/152875A1, WO2020/028537A1, WO2020/073047A1. Any of the anchors herein may comprises a deformable material, such as nitinol, and may be coupled to a scaffold section of the expandable pump portion. Coupled to in this context includes being integral with (e.g., unitary with) and being indirectly attached.

Anchors herein may have elongate finger or arm-like configurations, extending radially outward when deployed, such as exemplary anchors 120, 122, 124 and 126 shown in FIG. 1. Configurations in which they extend distally and radially relative to where they are coupled to the blood conduit, examples of which are shown in FIG. 1, may ease or facilitate the re-collapse and capture of the anchors when a sheath is advanced distally (relative motion) over the blood conduit and anchors.

The entirety of any of the anchors herein need not extend distally relative to the blood conduit to be able to facilitate collapse. For example, stabilizing members 124 may be considered to have a general curved, C, U, or bow shape when expanded, and initially extend radially and distally relative to the blood conduit, then extend further proximally at their radially outer ends as shown. When the sheath is advanced distally, stabilizing members 124 are adapted to be collapse and be re-sheathed. Additionally, any of the anchors herein may extend solely radially outward when expanded and will be able to be collapsed.

Any of the anchors herein may be separate components from the blood conduit (not unitary therewith), but may be secured to one or more other blood conduit components during manufacture. For example, any of the anchors herein may be secured to any portion of an expandable scaffold, examples of which are described in examples incorporated by reference herein. Anchors may optionally and alternatively be secured to and extend from a distal strut or a proximal strut of the expandable pump portion (not shown).

Anchors herein may be unitarily formed with one or more blood conduit components. For example, any of the anchors and scaffolds herein may be laser cut from the same nitinol tubular starting material. Forming the anchors unitarily with one or more blood conduit components may simplify construction by eliminating a step of coupling an anchor to a region of the blood conduit.

Any of the anchors herein may have distal free ends that are configured to further stabilize the anchor against tissue. For example, without limitation, any of the anchors herein may include an end that include one or more of barbs, protrusions, or any other type of element or configuration that is configured to increase the stability of the anchor with respect to the vessel wall, such as a descending aorta wall or IVC.

Any of the anchors herein may be equidistantly spaced apart around the shroud (circumferentially). For example, first and second anchors may be circumferentially spaced 180 degrees from each other, three anchors may be spaced 120 degrees from each other, four anchors may be spaced 90 degrees from each other, etc. In other embodiments, any of the anchors may not be circumferentially spaced equidistantly around the blood conduit.

FIG. 1 also shows exemplary distal expandable and collapsible struts 110, exemplary proximal expandable and collapsible struts 108, exemplary distal impeller 104, exemplary proximal impeller 106

Any of anchors herein may optionally be adapted to be inflatable, such as anchor 260′ and 260″ shown in the embodiment in FIG. 2. Any aspect of any other pump portion herein may be incorporated into the expandable pump portion shown in FIG. 2 (and vice versa), such as an expandable blood conduit, scaffold, membrane, impeller(s), and struts, examples of which are shown. Inflatable anchors 260′ and 260″ may be in fluid communication with inflation fluid in an inflation fluid source (not shown for clarity), which may be external to the patient. The pump portion in this embodiment has an inflatable anchor that comprises a distal inflatable anchor region 260″ and a proximal inflatable anchor region 260′, which are in fluid communication in this example with each other via an inflation line 270. Inflation lumen 260 is also in fluid communication with anchor region 260′ and extends along one of the proximal struts and then proximally through the catheter portion of the blood pump, as shown in FIG. 2.

Any of the blood pumps herein may include an inflation lumen that extends to or towards an external portion of the blood pump, such as an external console or other external control portion of the pump. The inflation lumen may be in communication with an inflation fluid source, such as a gas source or a liquid source. One or more external pumps may help control the inflation of inflation fluid to the inflatable anchor(s). Inflation of the one or more inflatable anchors may occur at any time after the pump portion is deployed from a delivery sheath, such as sheath 220 as shown in FIG. 2. Any the inflatable anchors herein may be coupled to the expandable blood conduit using a wide variety to bonding concepts, such as spot welding or other techniques to couple inflatable members to an expandable blood conduit.

Any of the inflatable anchors herein may be configured to allow for some blood flow around the pump portion, before, during, and/or after operation of the pump. For example, any inflatable anchor herein may have an at least partially helical configuration when inflated, such as the anchor regions 260′ and 260″ shown in FIG. 2, which can allow blood to continue to flow around the expandable blood conduit without the inflatable element completely occluding blood flow in the descending aorta or IVC. FIG. 2 also shows renal arteries 252.

Any of the inflatable anchors herein may be sized and configured such that when inflated and expanded with a fluid, they expand and engage the descending aorta wall 250 and help anchor the pump in place to minimize axial migration of the blood conduit during use.

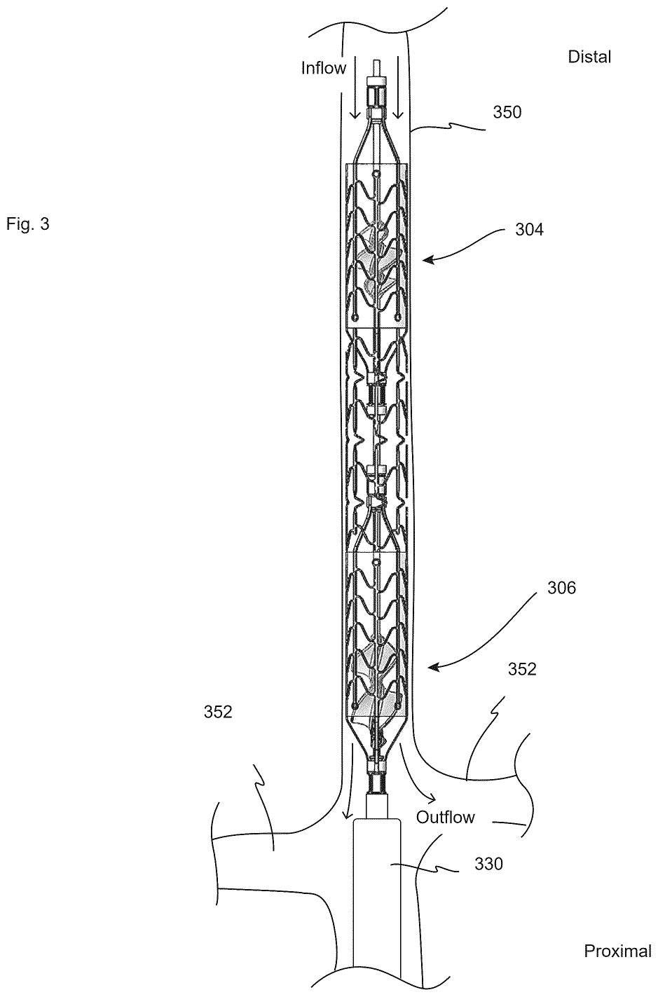

The outer dimension of any of the pump portions herein may be varied as needed for the implantation and/or the placement location within the patient. For example, the pump portion may be sized such that in an expanded configuration, an outer surface (optionally cylindrical) of the blood conduit directly engages or substantially engages a descending aorta or IVC sufficiently to anchor the pump in place and sufficiently minimize movement. An exemplary pump that is sized and configured to be expanded and anchored against a descending aorta 350 is shown in FIG. 3. The advantages of a multiple impeller pump apply in this embodiment, even if in this example the expandable pump portion is not collapsible to French sizes comparable to pump designs that have one or more anchors, for example. Any of the disclosure herein related to multi-impeller pumps is incorporated by reference into FIG. 3 and may be integrated into the example show in FIG. 3. FIG. 3 also illustrates exemplary distal impeller 304, proximal impeller 306, renal arteries 352 being perfused by the activated pump portion, and sheath 330 that can be used to deliver the expandable pump portion.

Exemplary descending aorta positions or locations for any of the pump portions herein are shown in FIGS. 1-3, with the pump portions positioned just upstream and proximate the ostia of the renal arteries, as shown. The position of the pump portions may be such that the proximal end of the blood conduit is aligned with, substantially aligned with, or just upstream, one or both of the ostia of the renal arteries. The outflow of the pump portions, as shown in FIGS. 1-3, may include a radial component. Positioning the pump just upstream to or aligned with one or both renal arteries may help direct blood towards or into the renal arteries, which may help perfuse the renal arteries and kidneys.

In alternative embodiments not shown in FIGS. 1-3, the multiple impeller pump portion is not expandable and collapsible. Non-expandable pumps may be positioned in the descending aorta as shown in any of FIGS. 1-3 just upstream to or aligned with a renal artery such that the pump outflow is still at least partially directed towards one or both renal arteries.

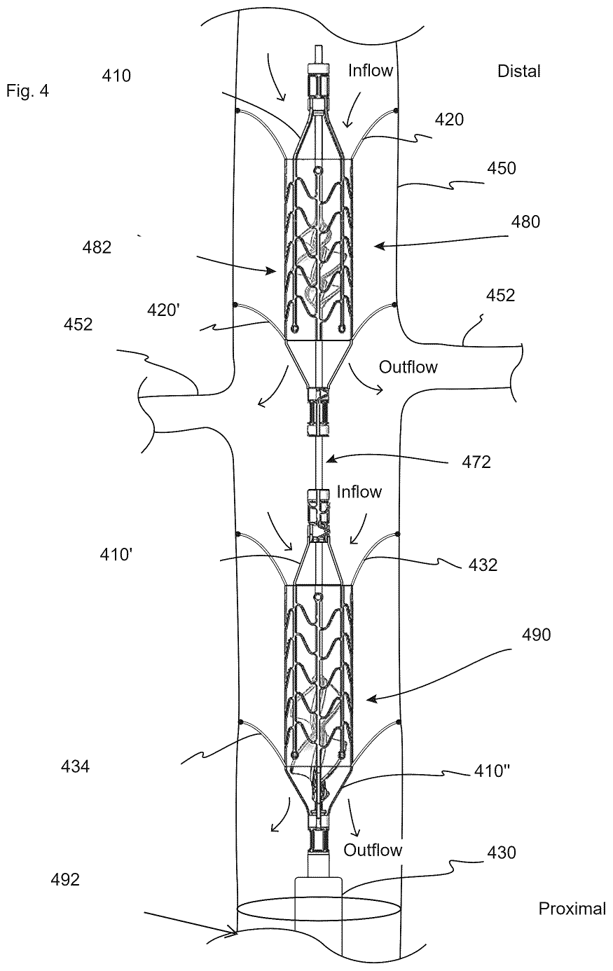

FIG. 4 illustrates an exemplary embodiment of a pump portion that is similar to the pump portions shown in FIG. 1-3, and applicable disclosure related to FIGS. 1-3 is incorporated by reference into the embodiment of FIG. 4. The blood pump shown expanded in place in FIG. 4 does not have a fluid conduit that extends all the way in between distal and proximal impellers, as shown. The pump portion in FIG. 4 includes a distal pump portion 480 that includes an expandable scaffold or basket, and a proximal pump portion 490 that includes an expandable scaffold or basket, each of which at least partially surrounds an impeller (distal impeller 482 is labeled). In this embodiment, the impellers may be rotated by the same or an otherwise common drive mechanism 472 (e.g., a drive cable or drive shaft), which may be rotated by an external motor that is also in rotational communication with the impellers. The distal pump portion 480 includes a distal blood conduit with an inflow and an outflow as shown, and the proximal pump portion 490 includes a proximal blood conduit with an inflow and an outflow as shown. The blood conduits may each include a scaffold coupled to a membrane. The central region between the distal pump portion 480 and the distal pump portion 490 is open and does not include a blood conduit, which allows blood pumped by the distal pump portion 480 to perfuse the renal arteries, as shown by the outflow of the distal pump portion 480. The proximal pump portion 490 pumps blood towards the lower region of the body, as shown. The distal pump portion 480 is shown positioned just upstream to the renal arteries 452, but the proximal end of the distal blood conduit may also be substantially aligned with one or more renal arteries so the outflow aids to perfuse one or both renal arteries. Exemplary anchors 420, 420′, 432 and 434 are also shown, which may be sized and configured to expand and contact the descending aorta 450 to anchor the pump portion in the ascending aorta.

The pump portion in FIG. 4 may alternatively or in addition to incorporate any suitable feature described with respect to the embodiments in FIGS. 1-3. For example, a variation of the pump shown in FIG. 4 includes one or more inflatable anchors, such as those shown in FIG. 2, to help anchor the pump portion in the descending aorta. Alternatively, the embodiment in FIG. 4 may be sized such that outer surfaces (e.g., cylindrical surfaces) of one or both of pump portions 480 and 490 expand into direct contact with descending aorta tissue 450, such as is the case in the embodiment shown in FIG. 3. This is an example of suitable features from any of the embodiments herein being combined with suitable features of any of the other embodiments herein.

In a variation on the blood pump shown in FIG. 4, a proximal portion of the distal impeller extends to some extent proximally beyond the proximal end of the distal blood conduit, as is shown with the relative positions of the proximal impeller in FIGS. 1-3. This may cause the outflow from the distal pump portion 480 to have more of a radial component than distal pump portions in which the distal impeller does not extend proximally beyond the proximal end of the blood conduit. This may help perfuse the renal arteries to a greater extent by directing more of the outflow from the distal pump portion radially outwards towards the ostia of the renal arteries.

In a variation of the blood pump of FIG. 4, the proximal pump portion 490 may include an impeller that is adapted and configured such that when the impeller is rotated, the impeller causes the proximal pump portion 490 to pump blood in a retrograde direction opposite that of the normal flow of blood, which is upward or in a superior direction in FIG. 4. In this variation, the proximal pump portion 490, when activated, pumps blood in a retrograde direction towards the ostia of the renal arteries rather than pumping blood downstream. In these variations, the impellers may be driven by a common drive mechanism 472 (e.g., a drive cable and/or drive shaft). In some methods of use, the operation of the impellers of this variation may be controlled in a manner related to the cardiac cycle of the heart. In some uses, for example, the impellers may be activated synchronously with the cardiac cycle. For example without limitation, the distal and proximal impellers can be turned on or activated during systole or a portion of systole (e.g., peak systole), and deactivated during diastole or a portion of diastole. Controlling pump operation in this exemplary manner may help perfuse the renal arteries during systole.

FIG. 4 also illustrates optional occluding member 492. Occluding member 492 may be controllably inflated and deflated to controllably occlude blood flow through the descending aorta in the vicinity of the pump portion in coordination with the cardiac cycle to help perfuse the kidneys. With respect to the original description of FIG. 4 in which both impellers pump blood in an antegrade direction when rotated, the blood pump may include an optional inflatable and deflatable occluding member 492. The occluding member may be coupled to sheath 430 as shown, but in alternative embodiments it may be secured to the catheter shaft. The inflatable and deflatable member 492 may be coupled to an outer surface of the sheath or the catheter shaft, and may be in fluid communication with a fluid source external to the patient via an inflation pathway extending through the catheter. In some embodiments, the occluding member 492 may be controllably inflated to occlude blood flow downstream the occluding member during systole to help perfuse the kidneys, and may be controllably deflated during diastole to allow blood to flow downstream.

An aspect of this disclosure is related to intravascular blood pumps with pump portions that are sized and configured for placement in the vicinity of the junction of an inferior vena cava (“IVC”) and one or more renal veins, and operated to reduce renal venous pressure. For example, in variations on FIGS. 1-4, the blood pump may instead be positioned on the venous side of the vasculature so the pump portion is delivered and deployed in an IVC. FIG. 1-4 may be modified such that the renal arteries are labeled as renal veins, the descending aorta is an IVC, and the natural blood would flow out of the renal veins, into the IVC, and in a superior direction back towards the heart.

FIG. 5 illustrates an exemplary pump portion 500 of an exemplary blood pump that may be include any suitable aspect of any of the blood pump described herein, and which may include any suitable structural component of any blood pump herein, including any of those shown in FIG. 1-4. For example, pump portion 500 may be sized to expand and contact at least a portion of an IVC wall 513. Additionally, the pump portion in the embodiment of FIG. 5 may include one or more anchoring/stabilizing members adapted to expand radially outward from the blood conduit and engage an IVC wall. FIG. 5 shows the pump portion 500 positioned in an IVC 513, with the distal impeller 504 positioned superior to the renal veins 511, and a proximal impeller 506 positioned inferior to the renal veins 511. Relative positions of kidneys is also labeled.

The impellers 504 and 506 are configured to pump blood from the pump inflow, through the expanded blood conduit or shroud 521, and toward the pump outflow, as shown. One or more of the drive shaft rotation direction or impeller configuration causes the blood to be pumped in this direction through the expandable blood conduit 521.

In this exemplary embodiment, as shown in FIG. 5, the blood pump includes one or more flow controllers 519 that are positioned, sized and configured to facilitate the flow of blood from outside the blood conduit and into the blood conduit. The pump portion 500 is expanded in a position such that the one or more flow controllers 519 (shown generally as circular or oval in this embodiment) are positioned generally at the location of the renal veins, such that blood flow out of the veins may enter into the blood pump fluid conduit through the one or more flow controllers. For example without limitation, any of the one or more flow controllers may comprise an opening, or in some embodiments any of the flow controllers herein may include a one-way valve (e.g., check valves) that allow blood to pass into the blood conduit but not out of the blood conduit. Other types of suitable flow controllers may of course be incorporated into the pump portions herein. One or more flow controllers may be axially spaced (along the length of the pump portion) from other flow controllers. Flow controllers may be disposed circumferentially around a periphery of the blood conduit at any distance apart. For example, one or more flow controllers may be spaced 30 degrees, 60 degrees or 90 degrees apart. In any of the blood pumps herein, one or more flow controllers may not be circumferentially or axially spaced at regular intervals. In any of these embodiments, there may be from one-twenty separate flow controllers, such as from two to ten. FIG. 5 also shows exemplary delivery sheath 530.

In an alternative to that shown in FIG. 5, a pump portion with multiple impellers may be expanded in place at a location that is superior to both veins, similar to the superior placement of the pump portion relative to the renal arteries shown in FIG. 1. In these alternatives, the blood conduit would be downstream the renal veins, as opposed to only a portion of the pump portion being downstream as shown in FIG. 5.

With respect to any of the methods herein that position a pump portion in the arterial vasculature, such as in the embodiments in FIGS. 1-4, the blood pump may be percutaneously and trans-luminally delivered to a portion of the descending aorta or renal artery of the patient via a femoral artery of the patient, for example, although other known entry locations and access pathways may also be used to deliver the pump portion to the target anatomy.

With respect to any of the methods herein that position a pump portion in the venous vasculature, such as in the embodiments in FIG. 5, the blood pump may be percutaneously and trans-luminally delivered to a portion of the IVC or renal vein of the patient via a femoral vein, for example, although other known entry locations and access pathways may also be used to deliver the pump portion to the target anatomy, such as via a subclavian vein or jugular vein, for example. It is understood that the terms distal and proximal are used herein as is common to refer to the relative directions based on the pathway in which the blood pump is advanced, examples of which are shown in FIGS. 1-5.

In alternative embodiments, any of the pump portions herein may be positioned in a renal artery rather than in a descending aorta. For example, the pump portion may be delivered via a femoral artery and then advanced into a renal artery, optionally over a guidewire that has been previously advanced into a right or left renal artery, Similarly, any of the pump portions herein may be positioned in a renal vein rather than in an IVC. For example, the pump portion may be delivered via a femoral vein and then advanced into a renal vein, optionally over a guidewire that has been advanced into a right or left renal vein. In any of these embodiments, pump portions from separate blood pumps can be positioned bilaterally in first and second renal arteries, or in first and second renal veins, or in any combination of renal arteries and renal veins (e.g., up to four pump portions delivered through separate sheaths). In any of these alternative embodiments, a pump portion positioned in a renal vein or a renal artery may optionally be a single impeller pump rather than a multiple impeller pump.

1. (canceled)

2. A circulatory assist device, comprising:
a pump portion having a distal end and a proximal end, having an expandable and collapsible blood conduit with a distal end and a proximal end between the distal and proximal ends of the pump portion;
a proximal expandable impeller, wherein at least a portion of the proximal impeller is disposed at the proximal end or a proximal end region of the blood conduit; wherein the blood conduit comprises a scaffold with a configuration that provides radial support to the blood conduit at the location of the proximal impeller;
one or more expandable anchors having deployed configurations that are sized and positioned to engage a descending aorta wall when the blood conduit is positioned in a descending aorta.

3. The device of claim 2, wherein at least one of the one or more expandable anchors is adapted to self-deploy when released from an outer sheath.

4. The device of claim 2, wherein at least one of the one or more expandable anchors is disposed at a distal end of the blood conduit.

5. The device of claim 2, wherein at least one of the one or more expandable anchors is disposed at the proximal end or a proximal end region of the blood conduit.

6. The device of claim 2, wherein at least one of the one or more expandable anchors is disposed between the distal end and the proximal end of the blood conduit.

7. The device of claim 2, wherein at least one of the one or more anchors is inflatable and is in fluid communication with an inflation fluid source.

8. The device of claim 7, wherein the at least one anchor that is inflatable is configured to allow blood flow around the blood conduit when inflated.

9. The device of claim 8, wherein the at least one anchor has an at least partially helical configuration.

10. The device of claim 7, wherein the at least one anchor has a distal expanded region and a proximal expanded region axially spaced from the distal expanded region, wherein the distal and proximal expanded regions are in fluid communication.

11. The device of claim 2, wherein the device further comprises a distal expandable impeller axially spaced from the proximal expandable impeller.

12. The device of claim 11, wherein the distal expandable impeller is disposed at the distal end or a distal end region of the blood conduit.

13. The device of claim 2, wherein the proximal expandable impeller is configured to produce a radial flow component for a fluid flowing out of the proximal end of the pump portion.

14. A circulatory assist device, comprising:
a pump portion comprising a proximal expandable blood conduit having a distal inflow and a proximal outflow; and
a proximal impeller disposed at least partially within the proximal expandable blood conduit when the proximal impeller is in an expanded configuration.

15. The device of claim 14, wherein the proximal expandable blood conduit includes an expandable scaffold.

16. The device of claim 14, wherein a proximal portion of the proximal impeller extends proximally out of the proximal blood conduit.

17. The device of claim 14, further comprising one or more deformable proximal struts and one or more deformable distal struts extending axially from the proximal blood conduit.

18. The device of claim 14, wherein the pump portion further comprises a distal expandable blood conduit with a distal inflow and a proximal outflow; and a distal impeller disposed at least partially within the distal expandable blood conduit when the distal impeller is in an expanded configuration.

19. The device of claim 17, wherein the distal expandable blood conduit comprises an expandable scaffold, and/or one or more deformable proximal struts and one or more deformable distal struts extending axially from the distal expandable blood conduit.

20. The device of claim 17, wherein a proximal portion of the distal impeller extends proximally out of the distal expandable blood conduit.

21. The device of claim 14, wherein the proximal impeller is configured to produce a radial flow component of the proximal outflow.
